(19) United States
(12) Patent Application Publication
(10) Pub. No.: US 2022/0176056 A1
(43) Pub. Date: Jun. 9, 2022

(54) MEDICINE DELIVERY DEVICES AND METHODS FOR THE USE THEREOF

(71) Applicant: Zewski Corporation, Magnolia, TX (US)

(72) Inventors: Lawrence KILISZEWSKI, The Woodlands, TX (US); Arin N. HOLECEK, Colorado Springs, CO (US); Francisco D. SALDANA, Houston, TX (US); Kunal S. BIJLANI, Houston, TX (US); Athena Jiunshi CHIEN, San Mateo, CA (US); Rithika Reddy PRODDUTOOR, Frisco, TX (US); Samantha Lee McCLENDON, Marietta, GA (US); Franklin Daniel BRIONES, Kissimmee, FL (US); Hope Kiyomi FA-KAJI, Berkeley, CA (US); Pujita Sai Reddy MUNNANGI, Fremont, CA (US)

(21) Appl. No.: 17/210,199

(22) Filed: Mar. 23, 2021

Related U.S. Application Data
(60) Provisional application No. 63/146,275, filed on Feb. 5, 2021.
(60) Provisional application No. 63/123,352, filed on Dec. 9, 2020.

(51) Int. Cl. A61M 16/06; A61M 15/00; A61M 16/20; A61M 16/00

(52) U.S. Cl. CPC A61M 16/0666; A61M 15/0091; A61M 16/204; A61M 2205/50; A61M 2016/0018; A61M 2016/0027; A61M 2205/3553; A61M 16/024

(57) ABSTRACT

The present disclosure relates to medicine delivery devices, as well as methods of delivering an aerosol medicine to a subject in need thereof. A benefit to the medicine delivery devices and methods herein can be the efficient delivery of an aerosol medicine to a subject in need thereof, thus helping to improve treatment outcomes, as well as avoiding wastage of expensive medicines. Additional benefits to the medicine delivery devices disclosed herein can be low cost, lightweight, compact, versatile, and simple to use devices useful for a wide range of patients and healthcare settings. Another benefit of the medicine delivery devices can be safer to use devices that can lower the risk of infection for patients and healthcare providers.

### CROSS-REFERENCE TO RELATED APPLICATIONS

This application claims the benefit of U.S. Provisional Application No. 63/123,352, filed on Dec. 9, 2020, and U.S. Provisional Application No. 63/146,275, filed on Feb. 5, 2021, each of which is incorporated by reference herein in its entirety.

### TECHNICAL FIELD

The present disclosure relates to medicine delivery devices, as well as methods of delivering an aerosol medicine to a subject in need thereof. A benefit to the medicine delivery devices and methods herein can be the efficient delivery of an aerosol medicine to a subject in need thereof, thus helping to improve treatment outcomes, as well as avoiding wastage of expensive medicines. Additional benefits to the medicine delivery devices disclosed herein can be low cost, lightweight, compact, versatile, and simple to use devices useful for a wide range of patients and healthcare settings. Another benefit of the medicine delivery devices can be providing a disposable or single-use device that can lower the risk of infection for patients and healthcare providers.

### BACKGROUND

Mechanical ventilation is widely used to assist breathing in patients who are not able to breathe properly on their own. Positive pressure breathing assistance systems, such as continuous positive airway pressure (“CPAP”) systems, are conventional for the treatment of respiratory disorders, such as COVID-19, in adults, as well as children and infants. Medicines are frequently delivered during mechanized breathing assistance to patients in need of such treatment for COVID-19, ongoing respiratory distress syndrome (RDS), and other respiratory ailments. Current devices for administering drugs to a patient while receiving assisted breathing treatment are subject to considerable challenges, including low efficiency of drug delivery, high complexity, and high cost. There remains a need for medicine delivery devices that can address these challenges.

### SUMMARY

Embodiments of a medicine delivery device are disclosed herein. In various embodiments, the medicine delivery device includes an aerosol medicine dispenser connected by an air flow system to a pressure valve, a breath sensor configured to monitor a breathing action of a subject, and a programmable control module configured to receive a signal from the breath sensor and configured to control the pressure valve, the aerosol medicine dispenser, or a combination thereof.

In various embodiments, the subject is a human, an infant, an unconscious patient, a cat, a dog, a horse, or a mammal. In certain embodiments, the breathing action includes an inhalation, an exhalation, or a combination thereof or a pattern thereof.

In certain embodiments of a medicine delivery device, the breath sensor includes a respiratory rate monitor, a chest strap respiratory rate monitor, a waistband respiratory rate monitor, ECG (electrocardiogram) leads, a pressure sensor connected to a subject interface, a pyroelectric polymer transducer, a pyroelectric polymer film, an electrically conductive rubber belt, or a combination thereof.

In certain embodiments, the medicine delivery device further includes a subject interface, wherein the subject interface is configured to connect to the air flow system, wherein the subject interface includes a nasal cannula, a face mask, a breathing tube, a medicine port, or a combination thereof.

In certain embodiments, the aerosol medicine dispenser comprises a medicine delivery controller connected to a dispensing opening of a medicine reservoir, wherein the medicine delivery controller is connected to the air flow system, and wherein the medicine delivery controller comprises a nebulizer, an aerosolizer, an atomizer, a pressurized metered dose inhaler, a vaporizer, a fan, a hopper, or a combination thereof.

In some embodiments, the air flow system comprises at least one air tube, at least one air pipe, at least one air path, or a combination thereof.

In certain embodiments, the medicine delivery device further includes at least one electrical connection, wherein the at least one electrical connection connects the programmable control module to the breath sensor, the programmable control module to the pressure valve, the programmable control module to the aerosol medicine dispenser, or a combination thereof.

In certain embodiments, at least one of the programmable control module, the breath sensor, the pressure valve, and the medicine delivery controller comprises a wireless transmitter, a wireless receiver, or a combination thereof.

In certain embodiments, the pressure valve is configured to connect to at least one pressure source. In some embodiments, the at least one pressure source optionally includes an air pump, an air tank, air tube, an airline, or a combination thereof.

In certain embodiments, the programmable control module comprises a microcontroller and a programmer interface. In some embodiments, the programmable control module comprises a microcontroller and a wireless transmitter, a wireless receiver, or a combination thereof; or both.

In certain embodiments, the aerosol medicine dispenser contains a medicine, wherein the medicine is a liquid, a powder, or a slurry. In certain embodiments, the medicine includes a surfactant, an asthma drug, an antiproteinase inhibitor, a prostacylin analog, a mucoactive drug, gamma interferon, an immunosuppressant, a monoclonal antibody, an interferon, an anti-virus gene therapeutic, a bronchodilator, a corticosteroid, an anti-infective agent, an aminoglycoside, an antifungal drug, a hormone, a pain control drug, and combinations thereof.

Embodiments of methods of delivering an aerosol medicine to a subject in need thereof are disclosed herein. In an embodiment, such a method includes: providing a medicine delivery device, wherein the medicine delivery device comprises an aerosol medicine dispenser connected by an air flow system to a pressure valve, and a breath sensor configured to monitor a breathing action of a subject, and a programmable control module configured to receive a signal from the breath sensor and configured to control the pressure valve and the aerosol medicine dispenser; monitoring the breathing action of the subject; programming the programmable control module to dispense an amount of medicine for a treatment frequency during a treatment duration; providing a subject interface; and connecting the subject interface to the subject or the air flow system or both. In certain embodiments, the method further includes connecting the pressure valve to a pressure source.

In certain embodiments, the method further includes: flowing a treatment volume of air through the air flow system by opening the pressure valve during subject inhalation; and dispensing the amount of medicine from the aerosol medicine dispenser into the air flow system during subject inhalation. In certain embodiments, the method further includes closing the pressure valve during subject exhalation.

In certain embodiments, the method further includes programming the programmable control module to dispense the amount of medicine once per a number of inhalations.

Medicine delivery device kits are disclosed herein. In an embodiment, such a kit includes: an air flow system, wherein the air flow system comprises a least one tube or air flow path; an aerosol medicine dispenser, wherein the aerosol medicine dispenser comprises a medicine delivery controller connected to a dispensing opening of a medicine reservoir, and the medicine delivery controller, dispensing opening, or a combination thereof is configured to fasten onto a dispensing end of the air flow system; a pressure valve, wherein the pressure valve is configured to fasten onto to a pressure end of the air flow system; and a programmable control module and a breath sensor, wherein the programmable control module is capable of communicating with the aerosol medicine dispenser and the pressure valve.

### BRIEF DESCRIPTION OF THE DRAWINGS

The foregoing summary, as well as the following detailed description of the embodiments, will be better understood when read in conjunction with the attached drawings. For the purpose of illustration, there are shown in the drawings some embodiments, which may be preferable. It should be understood that the embodiments depicted are not limited to the precise details shown. Unless otherwise noted, the drawings are not to scale.

FIG. 1 shows a schematic illustration of a medicine delivery device according to embodiments disclosed herein.

FIG. 2 shows a schematic illustration of a medicine delivery device according to embodiments disclosed herein.

FIG. 3 shows a schematic illustration of a medicine delivery device according to embodiments disclosed herein.

FIG. 4 shows a schematic illustration of a medicine delivery device according to embodiments disclosed herein.

FIG. 5 shows a schematic illustration of a medicine delivery device according to embodiments disclosed herein.

FIG. 6 shows a schematic illustration of a subject interface of a medicine delivery device according to embodiments disclosed herein.

FIG. 7 shows a schematic cutaway side view of an aerosol medicine dispenser of a medicine delivery device according to embodiments disclosed herein.

FIG. 8 shows a schematic cutaway side view of an aerosol medicine dispenser of a medicine delivery device according to embodiments disclosed herein.

FIG. 9 shows a schematic cutaway side view of an aerosol medicine dispenser of a medicine delivery device according to embodiments disclosed herein.

FIG. 10 shows a schematic illustration of an aerosol medicine dispenser of a medicine delivery device according to embodiments disclosed herein.

FIG. 11 shows a schematic illustration of an aerosol medicine dispenser of a medicine delivery device according to embodiments disclosed herein.

FIG. 12A shows a schematic illustration of an aerosol medicine dispenser of a medicine delivery device according to embodiments disclosed herein.

FIG. 12B shows a schematic cutaway side view of an aerosol medicine dispenser of a medicine delivery device according to an embodiment shown in FIG. 12A.

FIG. 12C shows a schematic cutaway side view of an aerosol medicine dispenser of a medicine delivery device according to an embodiment shown in FIGS. 10 and 11.

FIG. 13 shows a flow chart illustrating a method of delivering an aerosol medicine to a subject in need thereof, according to embodiments disclosed herein.

### DETAILED DESCRIPTION

Unless otherwise noted, all measurements are in standard metric units.

Unless otherwise noted, all instances of the words “a,” “an,” or “the” can refer to one or more than one of the word that they modify.

Unless otherwise noted, the phrase “at least one of” means one or more than one of an object. For example, “at least one of the programmable control module, the breath sensor, the pressure valve, and the medicine delivery controller comprises a wireless transmitter” means that one of the listed objects (e.g. breath sensor) contains a wireless transmitter; more than one the listed objects (e.g. multiple breath sensors) contains a wireless transmitter; one, two, or 3 of the listed objects (e.g. the breath sensor and the pressure valve) contains a wireless transmitter or any combination thereof; and/or each of the programmable control module, the breath sensor, the pressure valve, and the medicine delivery controller contains a wireless transmitter.

Unless otherwise noted, the term “about” refers to ±10% of the non-percentage number that is described, rounded to the nearest whole integer. For example, about 100 mm, would include 90 to 110 mm. Unless otherwise noted, the term “about” refers to ±5% of a percentage number. For example, about 20% would include 15 to 25%. When the term “about” is discussed in terms of a range, then the term refers to the appropriate amount less than the lower limit and more than the upper limit. For example, from about 100 to about 200 mm would include from 90 to 220 mm.

Unless otherwise noted, properties (height, width, length, ratio etc.) as described herein are understood to be averaged measurements.

Unless otherwise noted, the terms “provide”, “provided” or “providing” refer to the supply, production, purchase, manufacture, assembly, formation, selection, configuration, conversion, introduction, addition, or incorporation of any element, amount, component, reagent, quantity, measurement, or analysis of any method or system of any embodiment herein.

Unless otherwise noted, the term “aerosol” refers to a suspension of liquid or solid particles in air or a gas.

Unless otherwise noted, the term “medicine” refers to a drug, medicine, aerosol medicine, medicament or therapeutic agent.

Unless otherwise noted, the term “subject” refers to a patient, a human, an infant, or an animal, including mammals.

Patients undergoing treatment for respiratory disorders are frequently administered medicines while undergoing assisted breathing treatment, commonly during CPAP or oxygen supplementation. Many patients, including adults as well as children and infants, need additional drugs for treating ongoing respiratory distress syndrome (RDS). RDS is caused by many underlying problems, among them premature birth, pneumonia, and other respiratory diseases, including severe acute respiratory syndromes, most recently COVID-19.

Mechanically assisted breathing action can be accomplished using a conventional form of ventilation treatment typically used for respiratory disorders, including a positive pressure breathing system or ventilator, such as a continuous positive airway pressure (CPAP) system, a nasal CPAP (NCPAP) system, or a bilevel positive airway pressure (BiPaP) system. Such systems use a constant positive pressure from an air source, or a gas source containing oxygen, to keep the airways dilated during inhalation and supply the patient with oxygen. The air or gas is pressurized and supplied from the air source or gas source through an air flow system. The air flow system can be connected to a subject interface, which commonly includes nasal prongs or a nasal cannula, nasopharyngeal tubes, a face mask, or an endotracheal tube. Positive pressure ventilator systems maintain a continuous positive airway pressure by using a restrictive air outlet device, or a pressure valve. The pressure valve can be located before, at the patient interface, or beyond the patient interface in the airflow path. While the machine is in operation, the pressure valve allows air to flow normally through the airflow path, but when pressure is eliminated, the valve seals to prevent backflow, thus preventing moisture or oxygen from flowing into the machine from the airflow path.

Administering drugs while a patient is on a positive pressure ventilator machine can be challenging. The current devices that allow drug delivery during treatment are plagued by low efficiency in drug delivery to the patient. Data from current systems show in many cases that less than 15% of the dosed drug actually makes it to the patient's lungs. This low efficiency is due in part to pressure-assisted systems utilizing a constant positive pressure to keep the airways dilated and prevent their collapse. When medicines are administered by being pumped into the lungs, the constant positive pressure results in the medicine being pumped continuously, while the patient is exhaling as well as inhaling. This results in some of the medicine being blown out of a pressure valve in the system, thus wasting some of the drug. In the case of liquid medications that are pumped into the lungs, the continuous positive pressure can result in liquid building up in the lungs. Not only do these caveats present difficulties for the effective treatment of patients, but many of the drugs used in these treatments are quite expensive, thus greatly increasing costs from the need to use considerably more of the drug to achieve the desired dosage. These medicines are in liquid form which makes them more expensive to store, ship, and purchase.

Systems that time drug delivery to coincide with patient inhalation have been developed; such systems have been shown to be able to increase drug delivery to 40% or more of the dosed drug to the lungs. While helping to solve the problems with efficiency, however, the current devices that allow for delivery of drugs during pressure-assisted breathing treatment are also quite expensive. They are also large, cumbersome, and difficult to transport. Many such devices are also not all inclusive, meaning that several products must be separately purchased in order to achieve the patient treatment goals.

Currently available devices are also typically designed to be cleaned and re-used between patients. The re-use of such devices, as well as the necessity to remove and connect or re-connect several different parts of the system, increases the risk of infection for the patients and healthcare providers. Preventing infection transmission has always been a challenge in healthcare settings, but it is of even greater importance in the present day of global pandemics.

There remains a need for medicine delivery devices that can not only efficiently and safely administer drugs to a patient during treatment with a positive pressure ventilator, but that can provide low cost devices that are lightweight, compact in size, and simple to use.

The medicine delivery devices of the present disclosure can monitor patient breathing to dispense the medicine during patient inhalation and withhold the medicine during patient exhalation. This design can avoid wasting medicine, avoid clogging the lines with medicine, and avoid inadvertently filling the patient's lungs with liquid or solid particles. The medicine delivery devices of the present disclosure can be disposable, single use, or for single-patient use to reduce the risk of contaminating patients or medical staff, and avoids the need for costly and/or time consuming cleaning of the medical device between patients. The medicine delivery devices of the present disclosure can enable the delivery of inexpensive, easily stored and transported solid medicines, which can then be dispensed as a solid particle in a gas or dissolved in a liquid for dispensing as liquid droplet in a gas. The medicine delivery devices of the present disclosure can provide an advantageous design that is modular and makes use of low cost, commercially available components for providing greater access to medicine delivery systems in poor or remote areas. Embodiments of medicine delivery devices herein can provide an important benefit of suitability for nearly any patient in any healthcare setting, thus helping to reduce recovery times and improve patient outcomes.

### Medicine Delivery Devices of Various Embodiments

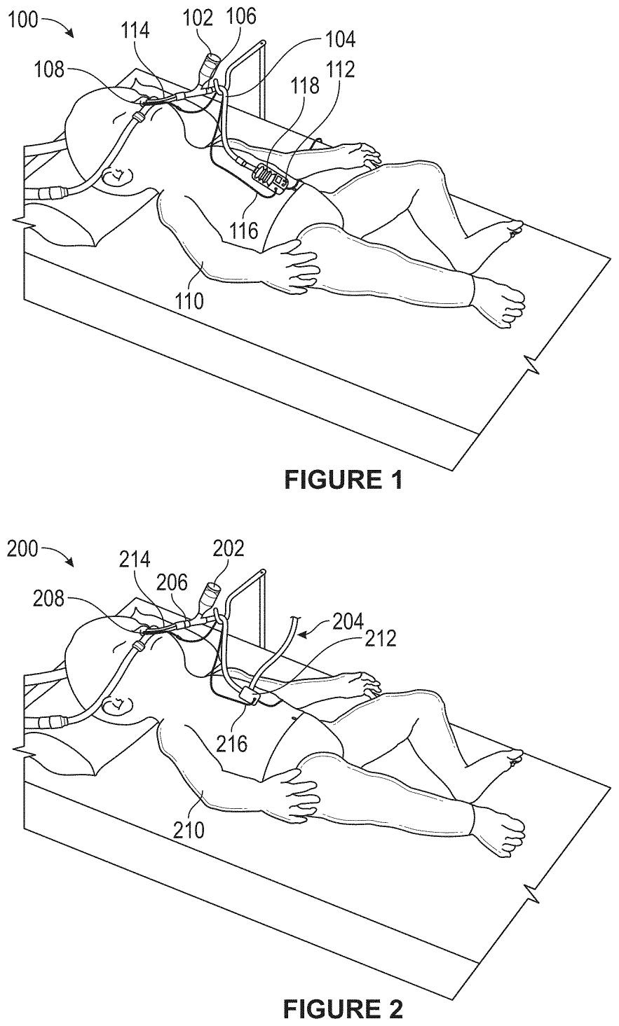

Embodiments of medicine delivery devices are disclosed herein. Referring to FIG. 1, medicine delivery device 100 includes aerosol medicine dispenser 102 connected by air flow system 104 to pressure valve 106; breath sensor 108 configured to monitor a breathing action of subject 110; programmable module 112 configured to receive a signal from breath sensor 108 and to control pressure valve 106; subject interface 114 configured to connect to air flow system 104; electrical connection 116 connecting programmable module 112 to breath sensor 108; and pressure source 118 configured to connect to pressure valve 106.

In an embodiment of a medicine delivery device, referring to FIG. 2, medicine delivery device 200 includes aerosol medicine dispenser 202 connected by air flow system 204 to pressure valve 206; breath sensor 208 configured to monitor a breathing action of subject 210; programmable module 212 configured to receive a signal from breath sensor 208 and to control pressure valve 206; subject interface 214 configured to connect to air flow system 204; and electrical connection 216 connecting programmable module 212 to breath sensor 208.

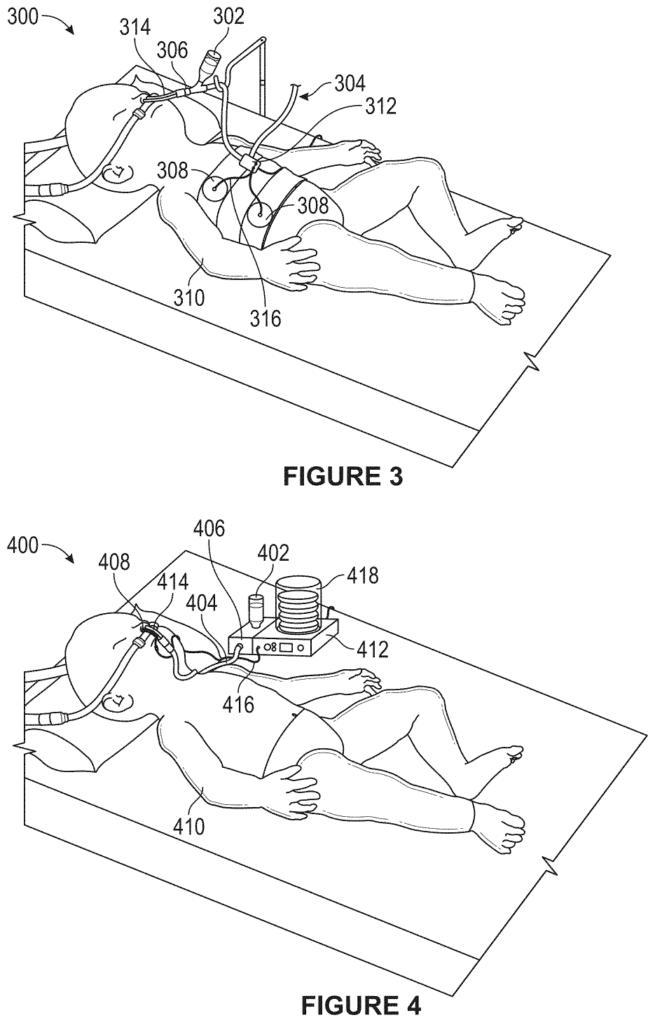

In an embodiment of a medicine delivery device, referring to FIG. 3, medicine delivery device 300 includes aerosol medicine dispenser 302 connected by air flow system 304 to pressure valve 306; breath sensors 308 configured to monitor a breathing action of subject 310; programmable module 312 configured to receive a signal from breath sensors 308 and to control pressure valve 306; subject interface 314 configured to connect to air flow system 304; and electrical connections 316 connecting programmable module 312 to breath sensors 308.

In an embodiment of a medicine delivery device, referring to FIG. 4, medicine delivery device 400 includes aerosol medicine dispenser 402 connected by air flow system 404 to pressure valve 406; breath sensor 408 configured to monitor a breathing action of subject 410; programmable module 412 configured to receive a signal from breath sensor 408 and to control pressure valve 406; subject interface 414 configured to connect to air flow system 404; electrical connection 416 connecting programmable module 412 to breath sensor 408; and pressure source 418 configured to connect to pressure valve 406.

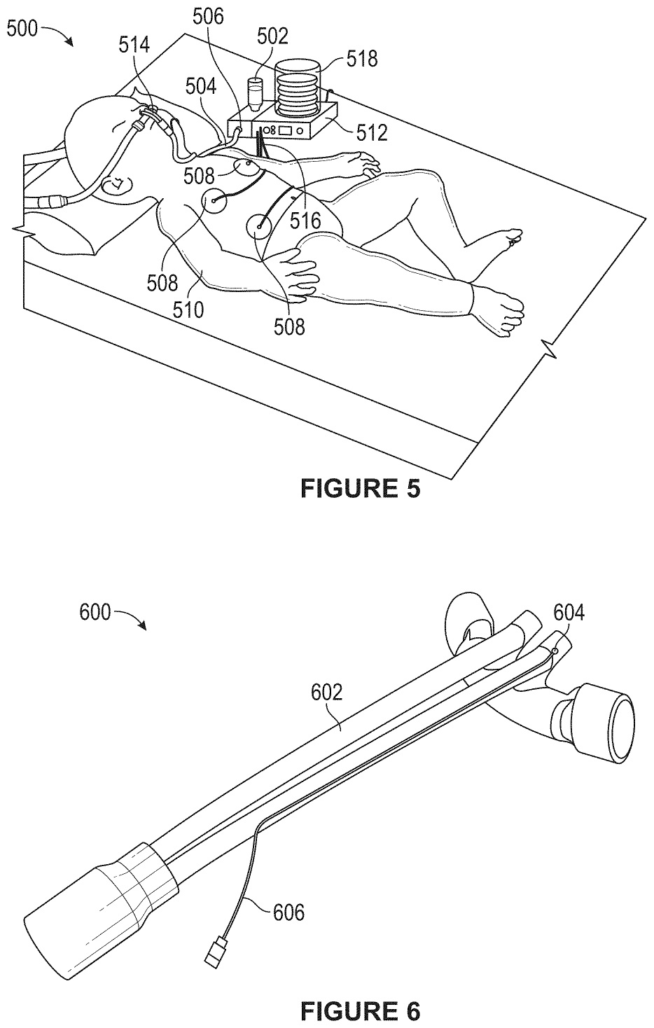

In an embodiment of a medicine delivery device, referring to FIG. 5, medicine delivery device 500 includes aerosol medicine dispenser 502 connected by air flow system 504 to pressure valve 506; breath sensors 508 configured to monitor a breathing action of subject 510; programmable module 512 configured to receive signals from breath sensors 508 and to control pressure valve 506; subject interface 514 configured to connect to air flow system 504; electrical connections 516 connecting programmable module 512 to breath sensors 508; and pressure source 518 configured to connect to pressure valve 506.

In an embodiment of a medicine delivery device, referring to FIG. 6, subject interface 600 includes nasal cannula 602, breath sensor 604, and electrical connection 606.

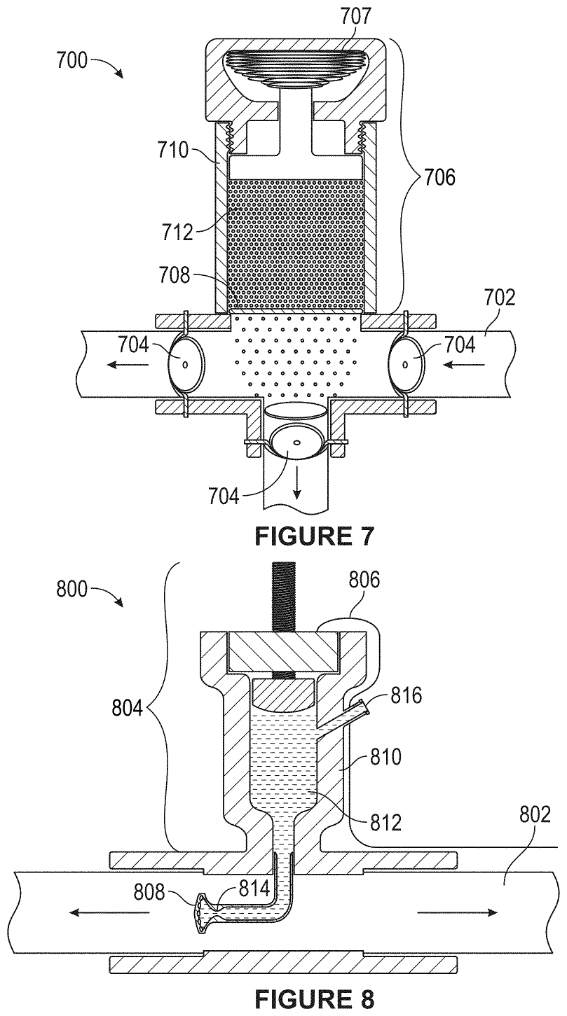

In an embodiment of a medicine delivery device, referring to FIG. 7, aerosol medicine dispenser 700 is connected by air flow system 702 to pressure valves 704, wherein aerosol medicine dispenser 700 includes medicine delivery controller 706 and actuator 707 connected to air flow system 702 and to dispensing opening 708 of medicine reservoir 710 containing medicine 712.

In an embodiment of a medicine delivery device, referring to FIG. 8, aerosol medicine dispenser 800 is connected to air flow system 802 and includes medicine delivery controller 804, electrical connection 806, dispensing opening 808 of medicine reservoir 810 containing medicine 812, and pressure valve 814. Medicine reservoir 810 includes medicine port 816.

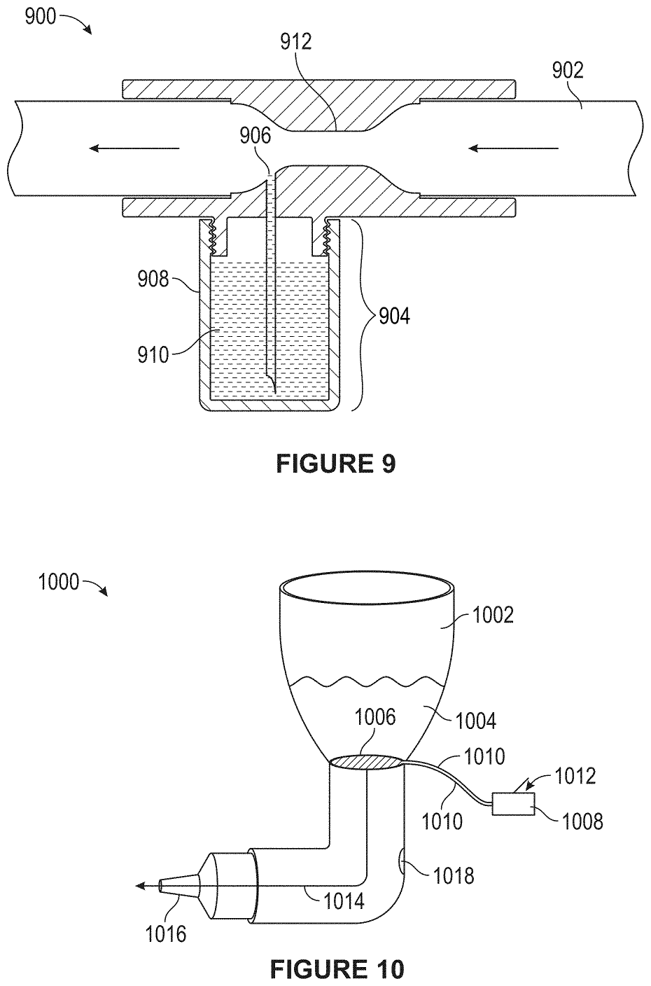

In an embodiment of a medicine delivery device, referring to FIG. 9, aerosol medicine dispenser 900 is connected to air flow system 902 and includes medicine delivery controller 904, dispensing opening 906 of medicine reservoir 908 containing medicine 910, and pressure valve 912.

In an embodiment of a medicine delivery device, referring to FIG. 10, aerosol medicine dispenser 1000 includes medicine reservoir 1002 containing medicine 1004 and connected to ultrasonic atomization disc 1006, atomization disc 1006 being connected to power source 1008 by electrical connections 1010 and controlled by switch 1012; aerosol flow path 1014 connected to ultrasonic atomization disc 1006 and subject interface connector 1016, and including air intake port 1018.

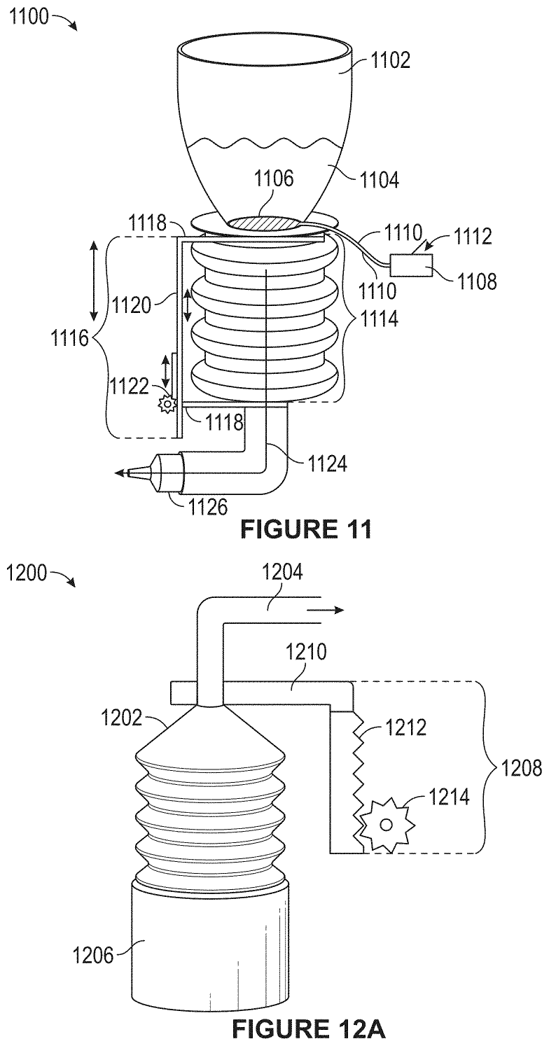

In an embodiment of a medicine delivery device, referring to FIG. 11, aerosol medicine dispenser 1100 includes medicine reservoir 1102 containing medicine 1104 and connected to ultrasonic atomization disc 1106, atomization disc 1106 being connected to power source 1108 by electrical connections 1110 and controlled by switch 1112; bellows 1114 connected to ultrasonic atomization disc 1106 and bellows actuator 1116, bellows actuator 1116 including compression arms 1118, vertical rack 1120, and gear 1122; and aerosol flow path 1124 connected to bellows 1114 and subject interface connector 1126.

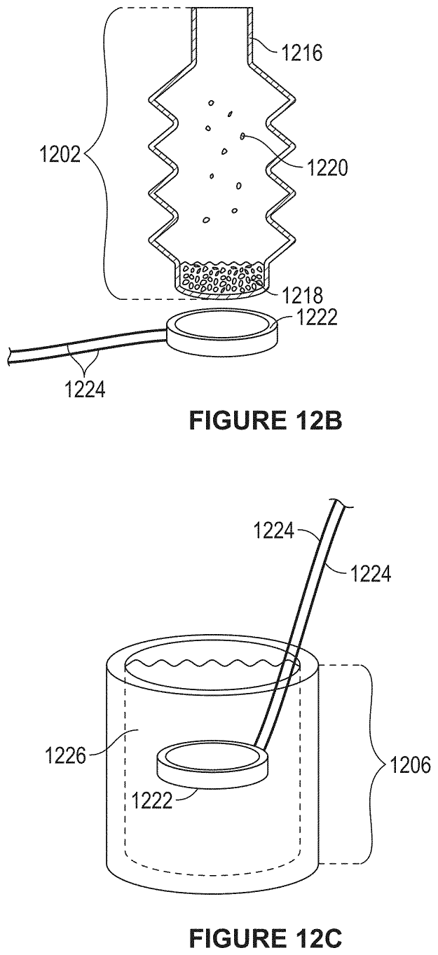

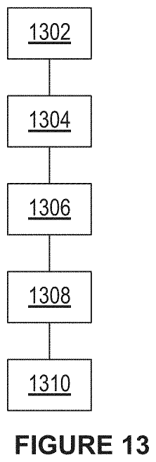

In an embodiment of a medicine delivery device, referring to FIG. 12A, aerosol medicine dispenser 1200 includes bellows 1202 connected to aerosol flow path 1204, water reservoir 1206, and bellows actuator 1208, bellows actuator 1208 including compression arm 1210, vertical rack 1212, and gear 1214.

In an embodiment of a medicine delivery device, referring to FIG. 12B, bellows 1202 includes flexible walls 1216 and contains medicine 1218 and medicine aerosol 1220, and is adjacent to ultrasonic atomization disc 1222 having electrical connections 1224.

In an embodiment of a medicine delivery device, referring to FIG. 12C, medicine reservoir 1206 contains medicine 1226 and ultrasonic atomization disc 1222 having electrical connections 1224.

Embodiments of a medicine delivery device are disclosed herein. In various embodiments, the medicine delivery device includes an aerosol medicine dispenser connected by an air flow system to a pressure valve, a breath sensor configured to monitor a breathing action of a subject, and a programmable control module configured to receive a signal from the breath sensor and configured to control the pressure valve, the aerosol medicine dispenser, or a combination thereof.

In various aspects, the breathing action of the subject can be a natural breathing action by the subject, or a mechanically assisted breathing action, or a combination thereof. Mechanically assisted breathing action can be accomplished using a conventional form of ventilation treatment typically used for respiratory disorders, including a positive pressure breathing assistance or ventilator system, such as a continuous positive airway pressure (CPAP) system, a nasal CPAP (NCPAP) system, or a bilateral positive airway pressure (BiPap) system. Air, or another oxygen gas mixture, can be supplied through the air flow system by an air or gas source.

The air flow system in various embodiments can include at least one air tube, at least one air pipe, at least one air path, or a combination thereof. In certain embodiments, the air flow system can include corrugated plastic tubing, a CPAP hose, or a rubber hose. The air flow system in various embodiments is connected to a subject interface. In various aspects, the subject interface can include a nasal cannula, a face mask, or a breathing tube. In certain embodiments, the airflow system can be connected to the subject interface using rubber or plastic connectors. In various embodiments, the subject is a human, an infant, an unconscious patient, a cat, a dog, a horse, or a mammal.

In various embodiments, the breathing action includes an inhalation, an exhalation, or a combination thereof or a pattern thereof. In various embodiments, the breath sensor is configured to monitor a breathing action of a subject. When operating the medicine delivery devices of various embodiments, during the monitoring of the breathing action, the breath sensor senses a breathing action in the subject, and sends a signal to the programmable control module; the programmable control module then controls the pressure valve, the aerosol medicine dispenser, or a combination thereof, to deliver an aerosol medicine dispensed from the aerosol medicine dispenser to the subject through the air flow system during the breathing action. In an aspect, the device can be configured to deliver an aerosol medicine during an inhalation by the subject. Such embodiments can have a benefit of improving the efficiency of delivery of a dose of aerosol medicine to the subject, by timing the medicine delivery to an inhalation by the subject. By targeting delivery of the medicine only during inhalation, the devices of various embodiments can result in more effective treatment of patients with aerosol medicines, thus improving patient outcomes. Such aspects that increase the efficiency of treatment can lead to a beneficial result of reducing the duration of patient hospital stays. Such embodiments can have a benefit of reducing the amount of an aerosol medicine that is required to effectively treat a patient, thus helping to avoid waste and to make more effective use of drugs that may be in short supply, as well as reducing costs, particularly considering the high cost of some drugs used to treat respiratory diseases.

The pressure valve of certain embodiments is configured to connect to at least one pressure source. In certain embodiments, the pressure source can be a pressure source that is incorporated into a positive pressure breathing assistance system. In certain embodiments, the pressure source can be an externally applied pressure source (see FIGS. 2-3). In certain embodiments, the pressure source can be a separate pressure source included in the medicine delivery device (see FIG. 1 and FIGS. 4-5). In certain embodiments, a pressure source included in the medicine delivery device can include an air pump. In certain embodiments, the pressure source can be a combination of that of a positive pressure breathing assistance system and a pressure source included in the medicine delivery device, or a combination of an externally applied pressure source and a pressure source included in the medicine delivery device. In some embodiments, the at least one pressure source optionally includes an air pump, an air tank, an air tube, an airline, or a combination thereof. In some embodiments, the pressure source, such as an air pump, can be connected to a programmable control module (see FIG. 1 and FIGS. 4-5). Embodiments wherein a separate pressure source is included in the medicine delivery device can provide a benefit of being separately controllable from the pressure source of the positive pressure breathing assistance system, thus adding to the versatility and utility of the device. Embodiments allowing direct control of the pressure source by the programmable control module of the medicine delivery device can also provide a benefit of greater accuracy of aerosol medicine delivery during an inhalation by the subject.

In certain embodiments, the medicine delivery device further includes a subject interface that is configured to connect to the air flow system. In certain embodiments, the subject interface includes a nasal cannula, a face mask, a breathing tube, a medicine port, or a combination thereof. Such embodiments including a subject interface in the medicine delivery device can have a benefit of avoiding the necessity of purchasing or supplying a separate subject interface for use of the device. Such embodiments can also provide a benefit of greater utility in use of the device for single patient use.

Various aspects of a medicine delivery device include a breath sensor. In certain embodiments, the breath sensor includes a respiratory rate monitor, a chest strap respiratory rate monitor, a waistband respiratory rate monitor, ECG leads, a pressure sensor connected to a subject interface, a pyroelectric polymer transducer, a pyroelectric polymer film, an electrically conductive rubber belt, or a combination thereof. In certain embodiments, more than one breath sensor of the same or a different type can be included in the medicine delivery device. Such embodiments can have a benefit of increasing the accuracy of the device to sense a breathing pattern of the subject, and thus increase the accuracy of the device to deliver an aerosol medicine during a desired breathing action. In an embodiment, the breath sensor includes a pressure sensor connected to a nasal cannula (see FIGS. 1-2 and 4). In such embodiments, the pressure sensor is connected to the nasal cannula at or near the area of the nasal cannula that inserts into the subject's nasal passages; a subject breathing action can then be detected by a pressure drop sensed by the pressure sensor. In some embodiments, the breath sensor can include one or more chest strap respiratory rate monitors, one or more waistband respiratory monitors, one or more ECG leads, one or more electrically conductive rubber belts, or a combination thereof (see FIGS. 3-5). In certain embodiments, a chest strap or a waist strap respiratory rate monitor can include an adjustable strap including a nylon or rubber material. In some embodiments, one of more breath sensors can include a wireless connection to the programmable control module of the medicine delivery device. Various embodiments of a breath sensor can provide a benefit of increasing the versatility of the device for use with a variety of different subjects and in various healthcare settings.

Various embodiments of a medicine delivery device include an aerosol medicine dispenser. Such a dispenser can include a bottle or another container, including a plastic or a glass material. In various aspects, the aerosol medicine dispenser is connected by an air flow system to a pressure valve. In certain embodiments, the pressure valve can include a one-way pressure valve or a spring-loaded valve. In certain embodiments, the pressure valve is connected to the airflow system between the aerosol medicine dispenser and the subject interface. In certain embodiments, the aerosol medicine dispenser is connected by an air flow system to a pressure valve by a connector, such as a T junction connector, a Y connector, a TY connector, a tube connector, or a tubing elbow. Such a connector can include a plastic or rubber material. In certain embodiments, the pressure valve is incorporated into the connector. In certain embodiments, the aerosol medicine dispenser can be connected to more than one pressure valve. In certain embodiments, the aerosol medicine dispenser can be connected to a programmable control module (see FIGS. 4-5). In certain embodiments, the aerosol medicine dispenser can be connected to the air flow system at or near the airflow connection to the subject interface; such embodiments can provide a benefit of a shorter travel distance of an aerosol medicine through the air flow system to be inhaled by the subject.

An aerosol medicine dispenser is disclosed herein. In certain embodiments, the aerosol medicine dispenser includes a medicine reservoir connected to an ultrasonic atomizer (see FIG. 10). In such embodiments, the ultrasonic atomizer can include an atomizer disc or fogger. In certain embodiments, the atomizer is connected to a power source by one or more electrical connections. Such a power source can include one or more switches, such as an on/off switch, to control the atomization disc. In some embodiments, the power source can be connected to a programmable control module or printed circuit board (PCB). In certain embodiments, the power source can be programmed to control the switch connected to the atomization disc, to turn the disc on or off, or to control an amount or rate of voltage supplied to the atomization disc. Such embodiments can provide benefits of controlling timing or the amount or rate of delivery of medicine to the subject through the air flow system, by controlling the action of the atomizer. In certain embodiments, an aerosol flow path is connected to the atomizer, wherein the aerosol flow path is connected to an air flow system and a subject interface. In certain embodiments, the aerosol flow path can be connected to a subject interface connector. Such a subject interface connector can include a plastic or rubber material. In certain embodiments, the aerosol flow path can include an air intake port. In certain embodiments, the air intake port can be connected to a solenoid valve that can be used to control the flow of air through the air intake port.

In certain embodiments, the aerosol medicine dispenser includes a medicine reservoir connected to an ultrasonic atomizer, and includes a bellows connected between the ultrasonic atomizer and an aerosol flow path (see FIG. 11). In certain embodiments, the action of the bellows can be controlled by a bellows actuator connected to the bellows. In such embodiments, the bellows actuator can include one or more compression arms connected to a top or a bottom portion of the bellows, the one or more compression arms being connected to a vertical rack. In certain embodiments, the one or more compression arms is controlled by one or more gears connected to the vertical rack. In certain embodiments, the one or more gears includes a motor driven gear; in such embodiments, the one or more gears can be connected to a motor shaft to allow a connected gear to rotate in a clockwise or counterclockwise direction. In certain embodiments of a motor driven gear, the gear can include a one-way bearing having an outer rotor, an inner rotor, and rollers to control the rotation of the gear in either direction. In certain embodiments, such a gear can rotate in a free flow direction but not rotate in the opposite or “no spin” direction. In such embodiments, in the no-spin direction, rollers move up the vertical rack and stop rotation via friction with the outer rotor. In such embodiments, the “no spin” direction can result in compression of the bellows, because the gear does not rotate along the vertical rack, while the motor shaft rotates. In the free flow direction, the gear rotates around a stationary motor shaft and moves along the vertical rack, resulting in expansion of the bellows. In such embodiments, the bellows actuator allows a unidirectional activation of the bellows. In certain embodiments, the free flow rotation is in a counterclockwise direction, and the no-spin direction is clockwise. In some embodiments, the bellows actuator can include a solenoid, worm-gear, or piston driven actuator.

In certain embodiments, the aerosol medicine dispenser includes a bellows connected to an aerosol flow path, a medicine reservoir, and a bellows actuator (see FIG. 12A). In certain embodiments, the aerosol medicine dispenser includes a bellows that also serves as a medicine reservoir (see FIG. 12B). In certain embodiments, the bellows includes flexible walls. Such flexible walls can include a plastic or a rubber material. In such embodiments, the bellows contains a medicine; in certain embodiments, the medicine is located in a bottom portion of the bellows, or adjacent to a flexible bottom wall of the bellows. In certain embodiments, the bellows can be pre-filled with a medicine, such as a liquid medicine, or filled with a medicine by an operator. In certain embodiments, an ultrasonic atomizer can be connected to or located in proximity to a portion of the bellows containing the medicine, such as a flexible bottom wall; in such embodiments, a medicine aerosol can be generated in the bellows by sonication or vibration of the atomization disc. Such embodiments can provide a benefit of allowing condensation generated on an inner surface of the bellows to be contained within the bellows by falling or draining back into the interior of the bellows. Such embodiments can provide benefits of avoiding waste of valuable drugs, as well as greater precision in medicine dosage. In certain embodiments, the atomizer is connected by one or more electrical connections to a power supply.

In certain embodiments, the aerosol medicine dispenser includes a water reservoir that can be connected to or located adjacent to a bellows that contains a medicine to be aerosolized (see FIG. 12C). In certain embodiments, the water reservoir is located adjacent to or connected to a bottom wall of the bellows, and in proximity to the medicine. In certain embodiments, an ultrasonic atomizer can be placed on a surface of water contained within the water reservoir and adjacent to or connected with the bellows. In certain embodiments, the atomizer is located adjacent to or connected to a bottom wall of the bellows, and in proximity to the medicine. In certain embodiments, the ultrasonic atomizer can be submerged in the water. Such embodiments can provide benefits of translation of sonication or vibrations from the water to the bellows, and provide increased heat capacity.

In certain embodiments, the aerosol medicine dispenser includes a medicine delivery controller connected to a dispensing opening of a medicine reservoir; in such embodiments, the medicine delivery controller is connected to the air flow system. In certain embodiments, the medicine delivery controller includes an aerosol generator. Such an aerosol generator can include a nebulizer, an aerosolizer, an atomizer, a pressurized metered dose inhaler, a vaporizer, a fan, a hopper, or a combination thereof. In certain embodiments, the medicine delivery controller includes a small particle aerosol generator, a small volume nebulizer, a valved holding chamber, or a dry powder inhaler. In certain embodiments, the aerosol medicine dispenser can include a medicine port to allow one or more medicines to be added to the medicine dispenser in the course of treatment.

In various aspects, the medicine delivery device further includes at least one electrical connection. In certain embodiments, the at least one electrical connection connects the programmable control module to the breath sensor, connects the programmable control module to the pressure valve, connects the programmable control module to the aerosol medicine dispenser, or a combination thereof. In certain embodiments, the at least one electrical connection includes one or more electric wires or cables; in such embodiments, the one or more electric wires or cables can include a sheath material including an electrically insulating material, a rubber material, a flame-retardant material, or a plastic material. In certain embodiments, the at least one electrical connection includes one or more reversible connectors located at a distal end of one or more electric wires, or in line with one or more electric wires. In certain embodiments, a reversible connector can include a USB plug, a cable jack, a coaxial power connector, a banana connector, a plug and socket connector, and a waterproof connector. Such embodiments including one or more reversible electric wire connections can provide benefits of convenience and versatility in connecting and disconnecting various elements of the medicine delivery device according to need. Such embodiments can also provide a benefit of decreasing the risk of the spread of infection by reducing the amount of handling of parts of the medicine delivery device that may become contaminated as a result of such handling.

In certain embodiments, at least one of the programmable control module, the breath sensor, the pressure valve, and the medicine delivery controller comprises a wireless transmitter, a wireless receiver, or a combination thereof. Such embodiments can provide benefits of versatility in the types of electrical connections between the various elements of the medicine delivery device, thus expanding the versatility of the device for use with a variety of different subjects and in various healthcare settings. Such embodiments can also have a benefit of mitigating the risk of the spread of infection by reducing the amount of handling of the device that is required during the course of treatment.

In various embodiments, the medicine delivery device is configured to use a power source. In certain embodiments, the power source includes an external power source, and the medicine delivery device includes at least one electrical connection configured to connect to the external power source. In some embodiments, the medicine delivery device includes at least one electrical connection that connects the programmable control module to an external power source. In an embodiment, the electrical connection can plug into an electrical wall outlet or other external power source. In some embodiments, the medicine delivery device includes an internal power source that is included in the medicine delivery device. In some embodiments, the medicine delivery device can use a combination of an external power source and an internal power source. In certain embodiments, the internal power source can include one or more batteries, or a battery pack. In certain embodiments, one or more batteries or a battery pack can be included in the programmable control module. In such embodiments, the one or more batteries or battery pack can be replaceable, or nonreplaceable. Embodiments wherein the medicine delivery device includes an internal power source can provide benefits of versatility for use of the device in healthcare settings, as well as greater utility of the device for single patient use.

In certain embodiments, the programmable control module includes a microcontroller and a programmer interface. In certain embodiments, the microcontroller is directly programmable via the programmer interface. In some embodiments, the programmable control module includes a microcontroller and a wireless transmitter, a wireless receiver, or a combination thereof; or both. Such embodiments can provide benefits of allowing the microcontroller to be remotely programmable, and increasing safety by reducing the requirement for close contact between a patient and a healthcare provider. The microcontroller in various embodiments can allow a healthcare provider to program the microcontroller to deliver desired volumes and rates of delivery of air, oxygen, and aerosol medicine, according to the prescribed treatment for the subject. For example, the microcontroller can be programmed to deliver a prescribed volume of air or oxygen per subject inhalation, or a prescribed amount of aerosol drug per subject inhalation, or a delivery of a prescribed amount of aerosol drug at a prescribed rate over a determined number of breathing actions, such as a dose delivery of drug after a determined number of subject inhalations. In certain embodiments, the delivery of air or oxygen is separately controllable by the microcontroller from the delivery of aerosol drug. The ability to separately program and control the air/oxygen delivery and aerosol drug delivery allows the healthcare provider to prevent the automatic delivery of aerosol drug with every inhalation, thus controlling the frequency of drug delivery and allowing the correct dosage to be supplied at the desired rate of delivery to the patient. In embodiments wherein the programmable control module controls the pressure valve, the aerosol medicine dispenser, or a combination thereof, the microcontroller can be programmed to sense inhalations and exhalations of a patient, and to control the pressure valve and the aerosol medicine dispenser to deliver an aerosol medicine dose timed with a patient inhalation.

In embodiments of a medicine delivery device herein, various components of the devices can be mostly, if not entirely, formed from a lightweight plastic material, or a cover or housing for various components can be formed from a lightweight plastic material. Such components can include, without limitation, the aerosol medicine dispenser, the air flow system, the pressure valve, the breath sensor, and the programmable control module. Such embodiments can provide benefits of low cost, lightweight, and compact devices that are simple to transport and store. Yet another benefit can be a utility of the devices for single patient use, so that a device can be used for a single patient and then entirely disposed of, without the need to clean or reuse any parts of the device.

In certain embodiments, the aerosol medicine dispenser contains a medicine, wherein the medicine is a liquid, a powder, or a slurry. In certain embodiments, the medicine includes a medicine that is capable of generating an aerosol in an aerosol generator. In certain embodiments, the medicine includes an aerosol drug formulation. In certain embodiments, the medicine includes a respiratory disease drug, and antiviral drug, or a COVID-19 drug. In certain embodiments, the medicine includes a surfactant, an asthma drug, an antiproteinase inhibitor, a prostacylin analog, a mucoactive drug, gamma interferon, an immunosuppressant, a monoclonal antibody, an interferon, an anti-virus gene therapeutic, a bronchodilator, a corticosteroid, an anti-infective agent, an aminoglycoside, an antifungal drug, a hormone, a pain control drug, and combinations thereof. In certain embodiments, the medicine includes a short-acting bronchodilator, albuterol sulfate, levalbuterol, ipratropium bromide, a long-acting bronchodilator, aclidinium bromide, arformoterol, formoterol, indacaterol, salmeterol, tiotropium, olodaterol, umeclidinium, glycopyrrolate, beclomethasone, budesonide, ciclesonide, flunisolide, fluticasone propionate, fluticasone furoate, mometasone furoate, dexamethasone, dornase alpha N-acetylcysteine, hyperosmolar saline, zanamivir, tobramycine, aztreonam, cromolyn sodium ribavirin, or mannitol.

### Embodiments of Methods of Delivering an Aerosol Medicine

Embodiments of methods of delivering an aerosol medicine to a subject in need thereof are disclosed herein. As an embodiment of a method disclosed herein, referring to FIG. 10, the method includes: providing a medicine delivery device 1002, wherein the medicine delivery device includes an aerosol medicine dispenser connected by an air flow system to a pressure valve, and a breath sensor configured to monitor a breathing action of a subject, and a programmable control module configured to receive a signal from the breath sensor and configured to control the pressure valve and the aerosol medicine dispenser; providing a subject interface 1004; connecting the subject interface to the subject or to the air flow system, or both 1006; monitoring the breathing action of the subject 1008; and programming the programmable control module to dispense an amount of medicine for a treatment frequency during a treatment duration 1010.

Embodiments of methods of delivering an aerosol medicine to a subject in need thereof are disclosed herein. In an embodiment, such a method includes: providing a medicine delivery device, wherein the medicine delivery device comprises an aerosol medicine dispenser connected by an air flow system to a pressure valve, and a breath sensor configured to monitor a breathing action of a subject, and a programmable control module configured to receive a signal from the breath sensor and configured to control the pressure valve and the aerosol medicine dispenser; monitoring the breathing action of the subject; programming the programmable control module to dispense an amount of medicine for a treatment frequency during a treatment duration; providing a subject interface; and connecting the subject interface to the subject or the air flow system or both. In certain embodiments, the method further includes connecting the pressure valve to a pressure source.

Various embodiments of a medicine delivery device herein can provide benefits that can also provide benefits for the use of such devices in embodiments of methods of delivering an aerosol medicine herein. Such benefits can include the efficient delivery of an aerosol medicine to a subject only during an inhalation phase of breathing, thus helping to improve treatment outcomes, as well as avoiding wastage of expensive medicines; the use of medicine delivery devices having a suitability and versatility for nearly any patient in any healthcare setting, thus helping to reduce recovery times and improve patient outcomes; and the use of medicine delivery devices that can mitigate the risk of the spread of infection by reducing the amount of handling of the device and patient contact required during the course of treatment.

In certain embodiments, the method further includes: flowing a treatment volume of air through the air flow system by opening the pressure valve during subject inhalation; and dispensing the amount of medicine from the aerosol medicine dispenser into the air flow system during subject inhalation. In certain embodiments, the method further includes closing the pressure valve during subject exhalation. Embodiments of a medicine delivery device wherein the programmable control module controls the pressure valve, the aerosol medicine dispenser, or a combination thereof, can provide a benefit of an ability of a healthcare provider to program the control module to sense inhalations and exhalations of a patient, and to control the pressure valve and the aerosol medicine dispenser to deliver an aerosol medicine dose timed with a patient inhalation.

In certain embodiments, the method further includes programming the programmable control module to dispense an amount of medicine once per a number of inhalations. Embodiments of medicine delivery devices herein can provide an ability to program a delivery of air or oxygen that is separately controllable by the microcontroller from the delivery of an aerosol drug. The ability to separately program and control the air/oxygen delivery and aerosol drug delivery allows the healthcare provider to prevent the automatic delivery of aerosol drug with every inhalation, thus controlling the frequency of drug delivery and allowing the correct dosage to be supplied at the desired rate of delivery to the patient.

### Medicine Delivery Kits of Various Embodiments

Medicine delivery device kits are disclosed herein. In an embodiment, such a kit includes: an air flow system, wherein the air flow system comprises a least one tube or air flow path; an aerosol medicine dispenser, wherein the aerosol medicine dispenser comprises a medicine delivery controller connected to a dispensing opening of a medicine reservoir, and the medicine delivery controller, dispensing opening, or a combination thereof is configured to fasten onto a dispensing end of the air flow system; a pressure valve, wherein the pressure valve is configured to fasten onto to a pressure end of the air flow system; and a programmable control module and a breath sensor, wherein the programmable control module is capable of communicating with the aerosol medicine dispenser and the pressure valve.

Various embodiments of a medicine delivery device herein can provide benefits that can also provide benefits for embodiments of medicine delivery kits. Such benefits can include low cost, lightweight, compact, simple to use devices for delivery of an aerosol medicine to a subject in need thereof. Another benefit of the medicine delivery kits can be providing safer to use devices that can lower the risk of infection for patients and healthcare providers. An additional benefit to the medicine delivery kits herein can be a packaging of the kits in sterile packaging, such as a sterile plastic or plastic lined paper packaging. Another benefit to the medicine delivery kits herein can be medicine delivery devices that include all parts needed in a variety of configurations that are useful for a broad range of different patients and healthcare settings, without the need to purchase additional parts. Yet another benefit to the medicine delivery kits can be a utility of the kits for single patient use, so that the kits can be used for a single patient and then entirely disposed of, without the need to clean or reuse any parts of the device.

### Examples

A pump was built to replicate the breathing of a subject. This simple disposable pump design was discovered to be adaptable to allow reversal of the breathing system and drug delivery, and to be adaptable for use in a device that would pump the drug, supplementing or replacing the function of the lungs. Wireless technology was also researched for adaptations for sensing breathing, core temperature, and various vital signs. Lastly in the development of the interface design, it was determined that the drug delivery is far more effective when automated and synchronized with the breathing pattern of the subject. These systems were combined to produce medicine delivery device designs. An Alpha prototype will be built using Arduino controllers and off the shelf components, while in parallel the system will be designed in CAD.

What is claimed is:

1. A medicine delivery device comprising:
an aerosol medicine dispenser connected by an air flow system to a pressure valve,
a breath sensor configured to monitor a breathing action of a subject, and
a programmable control module configured to receive a signal from the breath sensor and configured to control the pressure valve, the aerosol medicine dispenser, or a combination thereof.

2. The medicine delivery device of claim 1, wherein the breathing action includes an inhalation, an exhalation, or a combination thereof or a pattern thereof; or
wherein the subject is a human, an infant, an unconscious patient, a cat, a dog, a horse, or a mammal.

3. The medicine delivery device of claim 1, wherein the breath sensor comprises a respiratory rate monitor, a chest strap respiratory rate monitor, a waistband respiratory rate monitor, ECG leads, a pressure sensor connected to a subject interface, a pyroelectric polymer transducer, a pyroelectric polymer film, an electrically conductive rubber belt, or a combination thereof.

4. The medicine delivery device of claim 1, further comprising a subject interface,
wherein the subject interface is configured to connect to the air flow system,
wherein the subject interface includes a nasal cannula, a face mask, a breathing tube, a medicine port, or a combination thereof.

5. The medicine delivery device of claim 1, wherein the aerosol medicine dispenser comprises a medicine delivery controller connected to a dispensing opening of a medicine reservoir,
wherein the medicine delivery controller is connected to the air flow system, and
wherein the medicine delivery controller comprises a nebulizer, an aerosolizer, an atomizer, a pressurized metered dose inhaler, a vaporizer, a fan, a hopper, or a combination thereof.

6. The medicine delivery device of claim 1, wherein the air flow system comprises at least one air tube, at least one air pipe, at least one air path, or a combination thereof.

7. The medicine delivery device of claim 1, further comprising at least one electrical connection, wherein the at least one electrical connection connects the programmable control module to the breath sensor, the programmable control module to the pressure valve, the programmable control module to the aerosol medicine dispenser, or a combination thereof; or
wherein at least one of the programmable control module, the breath sensor, the pressure valve, and the medicine delivery controller comprises a wireless transmitter, a wireless receiver, or a combination thereof.

8. The medicine delivery device of claim 1, wherein the pressure valve is configured to connect to at least one pressure source, and
optionally, the at least one pressure source comprises an air pump, an air tank, air tube, an airline, or a combination thereof.

9. The medicine delivery device of claim 1, wherein the programmable control module comprises a microcontroller and a programmer interface; or
wherein the programmable control module comprises a microcontroller and a wireless transmitter, a wireless receiver, or a combination thereof; or both.

10. The medicine delivery device of claim 1, wherein the aerosol medicine dispenser contains a medicine, wherein the medicine is a liquid, a powder, or a slurry, and wherein the medicine includes a surfactant, an asthma drug, an antiproteinase inhibitor, a prostacylin analog, a mucoactive drug, gamma interferon, an immunosuppressant, a monoclonal antibody, an interferon, an anti-virus gene therapeutic, a bronchodilator, a corticosteroid, an anti-infective agent, an aminoglycoside, an antifungal drug, a hormone, a pain control drug, and combinations thereof.

11. A method of delivering an aerosol medicine to a subject in need thereof comprising:
providing a medicine delivery device, wherein the medicine delivery device comprises an aerosol medicine dispenser connected by an air flow system to a pressure valve, and a breath sensor configured to monitor a breathing action of a subject, and a programmable control module configured to receive a signal from the breath sensor and configured to control the pressure valve and the aerosol medicine dispenser;
monitoring the breathing action of the subject;
programming the programmable control module to dispense an amount of medicine for a treatment frequency during a treatment duration;
providing a subject interface; and
connecting the subject interface to the subject or the air flow system or both.

12. The method of claim 11, further comprising connecting the pressure valve to a pressure source.

13. The method of claim 11, further comprising:
flowing a treatment volume of air through the air flow system by opening the pressure valve during subject inhalation; and
dispensing the amount of medicine from the aerosol medicine dispenser into the air flow system during subject inhalation; and optionally,
closing the pressure valve during subject exhalation.

14. The method of claim 11, further comprising programming the programmable control module to dispense the amount of medicine once per a number of inhalations.

15. A medicine delivery device kit comprising:
an air flow system, wherein the air flow system comprises a least one tube or air flow path;
an aerosol medicine dispenser, wherein the aerosol medicine dispenser comprises a medicine delivery controller connected to a dispensing opening of a medicine reservoir, and the medicine delivery controller, dispensing opening, or a combination thereof is configured to fasten onto a dispensing end of the air flow system;
a pressure valve, wherein the pressure valve is configured to fasten onto to a pressure end of the air flow system; and
a programmable control module and a breath sensor, wherein the programmable control module is capable of communicating with the aerosol medicine dispenser and the pressure valve.
